(19) United States
(12) Patent Application Publication
(10) Pub. No.: US 2015/0196603 A1
(43) Pub. Date: Jul. 16, 2015

(54) Novel Cell Compositions and Methods

(71) Applicant: STEMNION, INC., Pittsburgh, PA (US)

(72) Inventors: Victoria Grammer, Pittsburgh, PA (US); Malcolm A Meyn, III, Pittsburgh, PA (US); Randall G. Rupp, Swanton, VT (US)

(73) Assignee: STEMNION, INC., Pittsburgh, PA (US)

(21) Appl. No.: 14/420,970

(22) PCT Filed: Aug. 2, 2013
(86) PCT No.: PCT/US13/53356

§ 371 Date: Feb. 11, 2015

Related U.S. Application Data
(60) Provisional application No. 61/776,031, filed on Mar. 11, 2013.
(60) Provisional application No. 61/683,896, filed on Aug. 16, 2012.

(51) Int. Cl. A61K 35/50

(52) U.S. Cl. CPC A61K 35/50

(57) ABSTRACT

The invention is directed to novel cell compositions termed AMP-N cells. In particular, the invention is directed to novel AMP-N cell compositions and novel compositions derived from the AMP-N cells including, but not limited to, novel cell-derived conditioned medium, termed ACCS-N. The invention is further directed to novel methods for making the novel AMP-N cell and ACCS-N compositions as well as uses thereof.

### CROSS-REFERENCE TO RELATED APPLICATIONS

This application claims priority under 35 USC §119(e) of U.S. Provisional Application No. 61/683,896, filed Aug. 16, 2012 and U.S. Provisional Application No. 61/776,031, filed Mar. 11, 2013, the entireties of which are incorporated herein by reference.

### STATEMENT REGARDING FEDERALLY SPONSORED RESEARCH OR DEVELOPMENT

This invention was made in part with United States government support awarded by the following agency: The Geneva Foundation, Subcontract no. S-1153-01, main contract no. W81XWH-08-2-0127. The United States may have certain rights to this invention.

### FIELD OF THE INVENTION

The field of the invention is directed to a novel cell composition termed AMP-N cells. In particular, the field of the invention is directed to the novel AMP-N cell composition and novel compositions derived from the AMP-N cells including, but not limited to, novel cell-derived conditioned medium, referred to herein as ACCS-N. The field of the invention is further directed to novel methods for making the novel AMP-N cell composition and novel ACCS-N composition as well as uses thereof.

### DESCRIPTION OF RELATED ART

Uchida S, et al. (2000) J Neurosci Res 62:585-590, describe the neurotrophic function of conditioned medium from human amniotic epithelial cells. Sheng, JG., et al. (1993) Exp Neurol 123(2):192-203, report on dopaminergic sprouting and behavioral recovery in hemi-parkinsonian rats after implantation of amnion cells. Lindvall, 0. and Kokaia, Z., (2006) Nature 441:1094-1096, describe progress in the use of stem cells for the treatment of neurological disorders. Kakishita, K., et al., (2000) Exp Neurol 165:27-34, report that human amniotic epithelial cells produce dopamine and survive after implantation into the striatum of a rat model of Parkinson's disease. Okawa, H., et al., (2001) Neuroreport 12(18):4003-7, report that amniotic epithelial cells transform into neuron-like cells in the ischemic brain. Kakishita, K., et al., (2003) Brain Res 980(1):48-56 describe implantation of human amniotic epithelial cells prevents the degeneration of nigral dopamine neurons in rats with 6-hydroxydopamine lesions. Meng, XT, et al., (2007) Cell Biol Int 31(7):691-8, report enhanced neural differentiation of neural stem cells and neurite growth by amniotic epithelial cell co-culture. Sankar, V. et al., (Neuroscience 2003, Letter to Neuroscience, 118(1):11-7) studied the role of human amniotic epithelial cell transplantation in transected spinal cord injury repair and reported that the human amniotic epithelial cells survived in monkey transected spinal cord, the graft was penetrated by host axons and there was no glial scar at the transection lesion site. Wu, Z-Y., et al, (Chinese Med Jour 2006, 119(24):2101-07) reported that transplantation of human amniotic epithelial cells improves hindlimb function in rats with spinal cord injury.

### BRIEF SUMMARY OF THE INVENTION

It is an object of the instant invention to provide a novel cell composition having a unique phenotype and function. These novel cells, referred to herein as AMP-N cells, are created by culturing AMP cells under novel conditions such that the AMP cells change and exhibit a unique phenotype as assessed by both cell surface and intracellular marker analysis. Specifically, the AMP-N cells have been shown to express a unique combination of markers, some of which are found on neurons, some of which are found on glial cells, and some of which are found on AMP cells (described below). These AMP-N cells represent the first time such a unique combination of markers has been described in one cell. It is also an object of the invention to provide novel AMP-N cell-derived compositions, in particular, conditioned medium derived from the AMP-N cells, referred to herein as N-conditioned medium or ACCS-N. This novel conditioned medium contains a unique combination of secreted protein factors. It is further an object of the invention to use the AMP-N cells or ACCS-N, either alone or in combination with each other and/or in combination with other agents, including active and/or inactive agents, to treat subjects afflicted with a nervous system disease, disorder or injury (described in detail elsewhere in the specification) such that improvement or recovery from the nervous system disease, disorder or injury occurs.

Accordingly, a first aspect of the invention is a composition comprising AMP-N cells.

In a specific embodiment, the AMP-N cells express high levels of SSEA-4, GFAP, α-S100 and low levels of B-tubulin III, MAP2, PSA-NCAM and NFM.

Another specific embodiment is a pharmaceutical composition comprising AMP-N cells. In another specific embodiment, the pharmaceutical composition of AMP-N cells is contained in an article of manufacture, wherein the article of manufacture comprises the pharmaceutical composition, packaging material, and instructions for use of the pharmaceutical composition.

A second aspect of the invention is a composition comprising ACCS-N.

A specific embodiment of this aspect is a pharmaceutical composition comprising ACCS-N.

In another specific embodiment of this aspect, the pharmaceutical composition of ACCS-N is contained in an article of manufacture, wherein the article of manufacture comprises pharmaceutical composition, packaging material, and instructions for use of the pharmaceutical composition to treat a nervous system disease, disorder or injury.

In another specific embodiment, the ACCS-N is formulated for sustained-release.

Another specific embodiment is one in which the ACCS-N is pooled ACCS-N.

A third aspect of the invention is a method for treating a nervous system disease, disorder or injury comprising administering to a subject a therapeutically effective dose of a composition selected from the group consisting of AMP-N cells, ACCS-N and pooled ACCS-N.

Other features and advantages of the invention will be apparent from the accompanying description, examples and the claims. The contents of all references, pending patent applications and issued patents, cited throughout this application are hereby expressly incorporated by reference. In case of conflict, the present specification, including definitions, will control.

Definitions

As used herein, the terms “a” or “an” means one or more; at least one.

As used herein, the term “adjunctive” means jointly, together with, in addition to, in conjunction with, and the like.

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein or an intracellular protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “Amnion-derived Multipotent Progenitor cell” or “AMP cell” means a specific population of cells that are epithelial cells derived from the amnion. AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: about 5-16 ng/mL for VEGF, about 3.5-4.5 ng/mL for Angiogenin, about 100-165 pg/mL for PDGF, about 2.5-2.7 ng/mL for TGFβ2, about 0.68 μg/mL for TIMP-1 and about 1.04 μg/mL for TIMP-2. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion-derived cells, from which AMP cells are isolated, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved and novel compositions and populations of cells.

As used herein, the term “AMP-N cell(s)” means a novel cell type that exhibits a unique combination of neuronal, glial, AMP cell, and stem cell markers.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “serum-free” when referring to certain compositions, growth conditions, culture media, etc. described herein, is meant that no animal-derived serum, including human derived serum, is used in the preparation, growth, culturing, expansion, storage or formulation of the composition or process.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such cells are subcultured, each round of subculturing is referred to as a passage. As used herein, “primary culture” means the freshly isolated cell population.

As used herein, the term “pooled” means a plurality of compositions that have been combined to create a new composition having more constant or consistent characteristics as compared to the non-pooled compositions.

As used herein, the term “differentiation” means the process by which cells become progressively more specialized.

As used herein, the term “differentiation efficiency” means the percentage of cells in a population that are differentiating or are able to differentiate.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium.

As used herein, the term “ACCS-N”, including “pooled ACCS-N”, means conditioned medium that has been derived from AMP-N cells. ACCS-N is comprised of a unique combination of secreted proteins and may be useful in treating nervous system diseases, disorders and injuries.

The term “physiological level” as used herein means the level that a substance in a living system is found and that is relevant to the proper functioning of a biochemical and/or biological process.

The term “therapeutically effective amount” means that amount of a therapeutic agent necessary to achieve a desired physiological effect (i.e. treat a nervous system disease, disorder or injury).

The term “lysate” as used herein refers to the composition obtained when cells, for example, AMP-N cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases. In some instances, it may be desirable to lyse the cells and retain the cellular membrane portion and discard the remaining portion of the lysed cells. In other instances, it may be desirable to retain both portions.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, growth and differentiation factors.

The term “transplantation” as used herein refers to the administration of a composition comprising cells, including a cell suspension or cells incorporated into a matrix or tissue, that are either in an undifferentiated, partially differentiated, or fully differentiated form into a human or other animal.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

As used herein, the term “co-administer” can include simultaneous or sequential administration of two or more agents. The two or more agents need not be administered by the same route.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The terms “parenteral administration” and “administered parenterally” are art-recognized and refer to modes of administration other than enteral and topical administration, usually but not necessarily by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral and intrasternal injection or infusion. Other routes of administration include intranasal and transmucosal.

The terms “sustained-release”, “extended-release”, “time-release”, “controlled-release”, “slow-release” or “continuous-release” as used herein means an agent, typically a therapeutic agent or drug, that is formulated to dissolve slowly and be released over time.

The terms “bioerodable” or “bioerosion” as used herein mean a combination of physical (i.e. dissolution) and chemical (i.e. chemical bond cleavage) processes that result in the breakdown of a substance.

The term “biodegradable” or “biodegradation” as used herein means a biological agent (i.e. an enzyme, microbe or cell) is responsible for the breakdown of a substance.

The terms “bioresporbable” or “bioabsorptable” as used herein mean the removal of a breakdown product by cellular activity (i.e. phagocytosis).

As used herein, the term “nanoparticle” means particles of less than 100 nm in diameter that exhibit new or enhanced size-dependent properties compared with larger particles of the same material.

“Neuron,” “neuronal cell” and “neural cell” (including neural progenitor cells and neural stem cells) are used interchangeably to refer to nerve cells, i.e., cells that are responsible for conducting nerve impulses from one part of the body to another. Most neurons consist of three distinct portions: a cell body which contains the nucleus, and two different types of cytoplasmic processes: dendrites and axons. Dendrites, which are the receiving portion of the neuron, are usually highly branched, thick extensions of the cell body. The axon is typically a single long, thin process that is specialized to conducts nerve impulses away from the cell body to another neuron or muscular or glandular tissue. Axons may have side branches called “axon collaterals.” Axon collaterals and axons may terminate by branching into many fine filaments called telodendria. The distal ends of telodendria are called synaptic end bulbs or axonal terminals, which contain synaptic vesicles that store neurotransmitters. Axons may be surrounded by a multilayered, white, phospholipid, segmented covering called the myelin sheath, which is formed by Schwann cells in the peripheral nervous system and oligodendrocytes in the central nervous system. Axons containing such a covering are “myelinated.” Neurons include sensory (afferent) neurons, which transmit impulses from receptors in the periphery to the brain and spinal cord and from lower to higher centers of the central nervous system. A neuron can also be motor (efferent) neurons which convey impulses from the brain and spinal cord to effectors in the periphery and from higher to lower centers of the central nervous system. Other neurons are association (connecting or interneuron) neurons which carry impulses from sensory neurons to motor neurons and are located within the central nervous system. The processes of afferent and efferent neurons arranged into bundles are called “nerves” when located outside the CNS or fiber tracts if inside the CNS.

“Neural tissue” or “nervous tissue” includes any tissue that comprises a neural cell or a nerve. In addition to neurons, other cells that may be present include one or more of oligodendrocytes, astrocytes, ependymal cells, microglial cells or Schwann cells.

As used herein the term “nervous system” means all cells and tissues that comprise the brain, spinal cord and peripheral nerves. The term “central nervous system” or “CNS” means the brain and/or spinal cord, and the term “peripheral nervous system” or “PNS” means all cells and tissues which comprise the peripheral nerves.

As used herein, “nervous system disease or disorder” means any condition that causes or results in a functional and/or physical deficit in the central and/or peripheral nervous system. As used herein, “central nervous system disease or disorder” or “CNS disease or disorder” means any condition that causes or results in a functional and/or physical deficit in the brain and/or spinal cord and “peripheral nervous system disease or disorder” or “PNS disease or disorder” means any condition that causes or results in a functional and/or physical deficit in the cells and tissues which comprise the peripheral nerves.

The term “brain injury” refers to any and all injuries of the brain and can be caused by fracture or penetration of the skull (i.e. a vehicle accident, fall, gunshot wound), neurotoxins, infections, tumors, metabolic abnormalities, or a closed head injury such as in the case of rapid acceleration or deceleration of the head (i.e. Shaken Baby Syndrome, blast), blunt trauma, concussions, and concussion syndrome.

The terms “astrocytes” and “astroglial cells” refer to a type of glial cell that become reactive and up-regulates intermediate filament (IF) proteins, such as glial fibrillary acid protein (GFAP) and vimentin (Vim), under pathological conditions or after transplantation in the brain and retina. “GFAP” refers to “glial fibrillary acid protein” which is one of the major intermediate filament proteins of mature astrocytes. It is used as a marker to distinguish astrocytes from other glial cells during development.

As used herein, the phrase “axonal growth” or “axon growth” refers to the elongation or extension of an axon of a neural cell. An axon can elongate for distances of microns to meters. Extension or elongation of an axon is also referred to as “regeneration” of the axon of a neural cell and may result in the reestablishment of nerve cell connectivity.

As used herein, “spinal cord injury” means an injury in which the axons or nerve fibers of the spinal cord are interrupted, generally by mechanical forces.

As used herein, the term “spinal cord scarring” refers to the recruitment and proliferation of glial cells to the site of spinal cord injury. The densely packed cells (primarily reactive astrocytes) and their secretions form a dense cellular plaque known as the glial scar. This scar prevents axons from projecting through thus interfering with axonal regeneration and functional recovery.

As used herein, the term “neuroprotection” means to arrest and/or reverse progression of neurodegeneration. As used herein, the term “neurodegeneration” means the progressive loss of neurons. This includes but is not limited to immediate loss of neurons followed by subsequent loss of connecting or adjacent neurons.

### FIGURE LEGENDS

FIG. 1 shows the results of Quantitative RT-PCR comparing the expression of 28 genes in AMP-N cells and undifferentiated AMP cells.

FIG. 2 shows the results of Quantitative RT-PCR comparing the expression of 28 genes in AMP-N cells and human neurons.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984,“Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Obtaining and Culturing of Cells

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and optionally e) further proliferation of the cells, optionally using additional additives and/or growth factors (i.e. recombinant human EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology, for example, human serum albumin. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human serum albumin added at at least 0.5% and up to 10%. In particular embodiments, the human serum albumin is from about 0.5% to about 2%. In a specific embodiment the human serum albumin is at 0.5%. The human serum albumin may come from a liquid or a dried (powder) form and includes, but is not limited to, recombinant human serum albumin, PLASBUMINO normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium , STEMLINE® T-cell expansion medium or STEMLINE® II hematopoietic stem cell expansion medium, OPTI-PRO™ serum-free culture medium, or DMEM culture medium, with human albumin (PLASBUMIN® normal human serum albumin) added up to amounts of 10%.

The invention further contemplates the use of any of the above basal media wherein animal-derived proteins are replaced with recombinant human proteins and animal-derived serum, such as BSA, is replaced with human serum albumin. In preferred embodiments, the media is serum-free in addition to being animal-free.

Optionally, other factors are used. In one embodiment, human epidermal growth factor (EGF) at a concentration of between 0-1 μg/mL is used. In a preferred embodiment, the human EGF concentration is around 10-20 ng/mL. All supplements are clinical grade.

Treatment of AMP Cells to Create Novel AMP-N Cells

AMP cells are treated according to a proprietary, multistep process that causes them to change their phenotype to a phenotype having characteristics of neuronal, glial and stem cells.

Generation of Conditioned Medium (ACCS-N) from AMP-N cells

The AMP-N cells of the invention can be used to generate ACCS-N. In one embodiment, the AMP-N cells are created as described herein and 1×10⁶ cells/mL are seeded into T75 flasks containing between 5-30 mL culture medium, preferably between 10-25 mL culture medium, and most preferably about 15 ml 10 mL culture medium. The cells are cultured and the ACCS-N is collected after approximately 28 days in culture.

Characterization of AMP-N Cells and ACCS-N

The N-AMP cells and ACCS-N are characterized by any assay familiar with skilled artisans to assess their phenotype and therapeutic potential. Such assays include, but are not limited to, PCR, antibody array, gene array, ELISA, mass spectrometry, cell-based activity assays, flow cytometry, animal models, etc.

Formulation, Dosage and Administration

The compositions of the invention can be prepared in a variety of ways depending on the intended use of the compositions. For example, a composition useful in practicing the invention may be a liquid comprising an agent of the invention, i.e. AMP-N cells and/or ACCS-N and/or pooled ACCS-N, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention may be viscous or muco-adhesive or both viscous and muco-adhesive.

Compositions comprising AMP-N cells and/or ACCS-N and/or pooled ACCS-N may be administered to a subject to provide various cellular or tissue functions, for example, to treat a nervous system disease, disorder or injury. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their use in treating a nervous system disease, disorder or injury or restoring a therapeutically important metabolic function. The compositions may also be administered to the recipient in one or more physiologically acceptable carriers. Carriers for the AMP-N cells may include but are not limited to solutions of phosphate buffered saline (PBS) or lactated Ringer's solution containing a mixture of salts in physiologic concentrations, basal culture medium and the like.

Preferably the liquid composition is aqueous. Alternatively, the composition can take form of an ointment. In a particular embodiment, the composition is an in situ gellable aqueous composition. Such a composition can comprise a gelling agent in a concentration effective to promote gelling upon contact with the body. Suitable gelling agents non-restrictively include thermosetting polymers such as tetra-substituted ethylene diamine block copolymers of ethylene oxide and propylene oxide (e.g., poloxamine 1307); polycarbophil; and polysaccharides such as gellan, carrageenan (e.g., kappa-carrageenan and iota-carrageenan), chitosan and alginate gums. The phrase “in situ gellable” includes not only liquids of low viscosity that can form gels, but also more viscous liquids such as semi-fluid and thixotropic gels that exhibit substantially increased viscosity or gel stiffness upon administration.

Aqueous compositions of the invention have physiologically compatible pH and osmolality. Typically these compositions incorporate means to inhibit microbial growth, for example through preparation and packaging under sterile conditions and/or through inclusion of an antimicrobially effective amount of an acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

The composition can comprise a depot formulation comprising an active agent for administration. The depot formulation comprises a composition of the invention (i.e. AMP-N cells and/or ACCS-N and/or pooled ACCS-N). The microparticles comprising the compositions can be embedded in a biocompatible pharmaceutically acceptable polymer or a lipid encapsulating agent. The depot formulations may be adapted to release all or substantially all of the active material over an extended period of time. The polymer or lipid matrix, if present, may be adapted to degrade sufficiently to be transported from the site of administration after release of all or substantially all of the active agent. The depot formulation can be liquid formulation, comprising a pharmaceutical acceptable polymer and a dissolved or dispersed active agent. Upon injection, the polymer forms a depot at the injections site, e.g. by gelifying or precipitating.

The composition can comprise a solid article that can be inserted or implanted in a suitable location in the disease or injury site, where the article releases the active agent. Release from such an article is preferably to the nervous system, with which the solid article is generally in intimate contact. Solid articles suitable for insertion or implantation generally comprise polymers and can be bioerodible or non-bioerodible. Bioerodible polymers that can be used in preparation of implants carrying a composition in accordance with the present invention include without restriction aliphatic polyesters such as polymers and copolymers of poly(glycolide), poly(lactide), poly(.epsilon.-caprolactone), poly(hydroxybutyrate) and poly(hydroxyvalerate), polyamino acids, polyorthoesters, polyanhydrides, aliphatic polycarbonates and polyether lactose. Illustrative of suitable non-bioerodible polymers are silicone elastomers.

Support matrices into which the AMP-N cells and/or ACCS-N and/or pooled ACCS-N can be incorporated or embedded include matrices which are recipient-compatible and which degrade into products which are not harmful to the recipient.

Natural and/or synthetic biodegradable matrices are examples of such matrices. Natural biodegradable matrices include plasma clots, e.g., derived from a mammal, collagen, fibronectin, and laminin matrices. Suitable synthetic matrix material must be biocompatible to preclude immunological complications. It must also be resorbable. The matrix should be configurable into a variety of shapes and should have sufficient strength to prevent collapse upon implantation. Recent studies indicate that the biodegradable polyester polymers made of polyglycolic acid fulfill all of these criteria (Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991)). Other synthetic biodegradable support matrices include synthetic polymers such as polyanhydrides, polyorthoesters, and polylactic acid. Further examples of synthetic polymers and methods of incorporating or embedding compositions into these matrices are also known in the art. See e.g., U.S. Pat. Nos. 4,298,002 and 5,308,701.

One of the advantages of a biodegradable polymeric matrix is that AMP-N cells and/or ACCS-N and/or pooled ACCS-N can be incorporated directly into the support matrix so that it is slowly released as the support matrix degrades in vivo. In addition to the AMP-N cells and/or ACCS-N and/or pooled ACCS-N, other factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, biologically active compounds which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., growth factors such as epidermal growth factor (EGF) and heparin binding epidermal growth factor like growth factor (HB-EGF), could be incorporated into the matrix or be provided in conjunction with the matrix. Similarly, polymers containing peptides such as the attachment peptide RGD (Arg-Gly-Asp) can be synthesized for use in forming matrices (see e.g. U.S. Pat. Nos. 4,988,621, 4,792,525, 5,965,997, 4,879,237 and 4,789,734).

In another example, the AMP-N cells and/or ACCS-N and/or pooled ACCS-N may be incorporated in a gel matrix (such as Gelfoam from Upjohn Company). A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures involving direct physical access to diseased or damaged tissue, all of the described forms of the AMP-N cells and/or ACCS-N and/or pooled ACCS-N delivery preparations are available options. These compositions can be repeatedly administered at intervals until a desired therapeutic effect is achieved.

The three-dimensional matrices to be used are structural matrices that provide a scaffold to guide the process of tissue healing and formation. Scaffolds can take forms ranging from fibers, gels, fabrics, sponge-like sheets, and complex 3-D structures with pores and channels fabricated using complex Solid Free Form Fabrication (SFFF) approaches. As used herein, the term “scaffold” means a three-dimensional (3D) structure (substrate and/or matrix). It may be composed of biological components, synthetic components or a combination of both. Further, it may be naturally constructed by cells or artificially constructed. In addition, the scaffold may contain components that have biological activity under appropriate conditions. The structure of the scaffold can include a mesh, a sponge or can be formed from a hydrogel.

The design and construction of the scaffolding to form a three-dimensional matrix is of primary importance. The matrix should be a pliable, non-toxic, injectable porous template for vascular ingrowth. The pores should allow vascular ingrowth. These are generally interconnected pores in the range of between approximately 100 and 300 microns, i.e., having an interstitial spacing between 100 and 300 microns, although larger openings can be used. The matrix should be shaped to maximize surface area, to allow adequate diffusion of nutrients, gases and growth factors. At the present time, a porous structure that is relatively resistant to compression is preferred, although it has been demonstrated that even if one or two of the typically six sides of the matrix are compressed, that the matrix is still effective to yield tissue growth.

The polymeric matrix may be made flexible or rigid, depending on the desired final form, structure and function. For repair of a defect, for example, a flexible fibrous mat is cut to approximate the entire defect then fitted to the surgically prepared defect as necessary during implantation. An advantage of using the fibrous matrices is the ease in reshaping and rearranging the structures at the time of implantation.

The invention also provides for the delivery of AMP-N cells and/or ACCS-N and/or pooled ACCS-N in conjunction with any of the above support matrices as well as amnion-derived membranes. Such membranes may be obtained as a by-product of the process described herein for the recovery of AMP cells, or by other methods, such as are described, for example, in U.S. Pat. No. 6,326,019 which describes a method for making, storing and using a surgical graft from human amniotic membrane, US 2003/0235580 which describes reconstituted and recombinant amniotic membranes for sustained delivery of therapeutic molecules, proteins or metabolites, to a site in a host, U.S. 2004/0181240, which describes an amniotic membrane covering for a tissue surface which may prevent adhesions, exclude bacteria or inhibit bacterial activity, or to promote healing or growth of tissue, and U.S. Pat. No. 4,361,552, which pertains to the preparation of cross-linked amnion membranes and their use in methods for treating burns and wounds. In accordance with the present invention, AMP-N cells and/or ACCS-N and/or pooled ACCS-N may be incorporated into such membranes.

One of skill in the art may readily determine the appropriate concentration, or dose, of the AMP-N cells and/or ACCS-N and/or pooled ACCS-N, for a particular purpose. The skilled artisan will recognize that a preferred dose is one which produces a therapeutic effect, such as treating a nervous system disease, disorder or injury, in a patient in need thereof. Of course, proper doses of the AMP-N cells and/or ACCS-N and/or pooled ACCS-N, will require empirical determination at time of use based on several variables including but not limited to the severity and type of disease, injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of disease, injury, disorder or condition being treated. In one embodiment, one dose is sufficient. Other embodiments contemplate, 2, 3, 4, or more doses.

The present invention provides a method of treating a nervous system disease, disorder or injury by administering to a subject AMP-N cells and/or ACCS-N and/or pooled ACCS-N, in a therapeutically effective amount. By “therapeutically effective amount” is meant the dose of AMP-N cells and/or ACCS-N and/or pooled ACCS-N which is sufficient to elicit a therapeutic effect. Thus, the concentration of AMP-N cells and/or ACCS-N and/or pooled ACCS-N in an administered dose unit in accordance with the present invention is effective in, for example, treating a nervous system disease, disorder or injury.

By way of non-limiting example, AMP-N cells are prepared at a concentration of between about 1×10⁷-1×10⁸ cells/mL, preferably at about 2.5×10⁷-7.5×10⁷ cells/mL, and most preferably at about 5×10⁷ cells/mL. The volume of cell mixture administered will depend upon several variables and can only be determined by the attending physician at time of use. Such proper doses of the AMP-N cells will require empirical determination based on such variables as the severity and type of disease, injury, disorder or condition being treated; patient age, weight, sex, health; other medications and treatments being administered to the patient; and the like.

In addition, one of skill in the art may readily determine the appropriate dose of the ACCS-N and/or pooled ACCS-N for a particular purpose. A preferred dose is in the range of about 0.1-to-1000 micrograms per square centimeter of applied area. Other preferred dose ranges are 1.0-to-50.0 micrograms/applied area. In a particularly preferred embodiment, it is expected that relatively small amounts of the ACCS-N and/or pooled ACCS-N will be therapeutically useful. One of skill in the art will also recognize that the number of doses to be administered needs also to be empirically determined based on, for example, severity and type of disease, disorder or injury being treated. For example, in a preferred embodiment, one dose is sufficient to have a therapeutic effect. Other preferred embodiments contemplate, 2, 3, 4, or more doses for therapeutic effect.

In further embodiments of the present invention, it may be desirable to co-administer other agents, including active agents and/or inactive agents, with the AMP-N cells and/or ACCS-N and/or pooled ACCS-N to treat a nervous system disease, disorder or injury. Active agents include but are not limited to cytokines, chemokines, antibodies, inhibitors, antibiotics, anti-fungals, anti-virals, immunosuppressive agents, other cell types, and the like. Inactive agents include carriers, diluents, stabilizers, gelling agents, delivery vehicles, ECMs (natural and synthetic), scaffolds, matrices, nanoparticles and the like. When the AMP-N cells and/or ACCS-N and/or pooled ACCS-N are administered conjointly with other pharmaceutically active agents, even less of the AMP-N cells and/or ACCS-N and/or pooled ACCS-N may be needed to be therapeutically effective.

AMP-N cells and/or ACCS-N and/or pooled ACCS-N can be administered by injection into a target site of a subject via a delivery device, such as a tube, catheter, syringe, needle, atomizer, nebulizer, and the like, through which the AMP-N cells and/or ACCS-N and/or pooled ACCS-N can be introduced into the subject at a desired location.

Routes of administration include enteral, topical, intranasal, transmucosal, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral and intrasternal administration. The appropriate route of administration will depend upon the disease, disorder, injury and site being treated.

The timing of administration of AMP-N cells and/or ACCS-N and/or pooled ACCS-N will depend upon the type and severity of the nervous system disease, disorder or injury being treated. In a particular embodiment, the AMP-N cells and/or ACCS-N and/or pooled ACCS-N are administered as soon as possible after the a nervous system disease, disorder or injury is diagnosed. In other particular embodiments, the AMP-N cells and/or ACCS-N and/or pooled ACCS-N are administered more than one time following injury or diagnosis.

Sustained-Release Compositions

The AMP-N cells and/or ACCS-N and/or pooled ACCS-N may be formulated as sustained-release compositions. Skilled artisans are familiar with methodologies to create sustained-release compositions of therapeutic agents, including protein-based therapeutic agents such as AMP-N cells and/or ACCS-N and/or pooled ACCS-N.

The sustained-release compositions may be made by any of the methods described herein. For example, multivesicular liposome formulation technology is useful for the sustained-release of protein and peptide therapeutics. Qui, J., et al, (ACTA Pharmacol Sin, 2005, 26(11):1395-401) describe this methodology for the formulation of sustained-release interferon alpha-2b. Vyas, S. P., et al, (Drug Dev Ind Pharm, 2006, 32(6):699-707) describe encapsulating pegylated interferon alpha in multivesicular liposomes. AMP-N cells and/or ACCS-N and/or pooled ACCS-N are suitable for use in multivesicular liposome sustained-release formulations.

Nanoparticle technology is also useful for creating sustained-release compositions. For example, Packhaeuser, C. B., et al, (J Control Release, 2007, 123(2):131-40) describe biodegradable parenteral depot systems based on insulin loaded dialkylaminoalkyl-amine-poly(vinyl alcohol)-g-poly(lactide-co-glycolide) nanoparticules and conclude that nanoparticle-based depots are suitable candidates for the design of controlled-release devices for bioactive macromolecules (i.e. proteins). Dailey, L. A., et al, (Pharm Res 2003, 20(12):2011-20) describe surfactant-free, biodegradable nanoparticles for aerosol therapy which is based on the branched polymers DEAPA-PVAL-g-PLGA and conclude that DEAPA-PVAL-g-PLGA are versatile drug delivery systems. AMP-N cells and/or ACCS-N and/or pooled ACCS-N are suitable for use in nanoparticle-based sustained-release formulations.

Polymer-based sustained-release formulations are also very useful. Chan, Y. P., et al, (Expert Opin Drug Deliv, 2007, 4(4):441-51) provide a review of the Medusa system (Flamel Technologies), which is used for sustained-release of protein and peptide therapies. Thus far, the Medusa system has been applied to subcutaneous injection of IL-2 and IFN-alpha(2b), in animal models (rats, dogs, monkeys), and in clinical trials in renal cancer (IL-2) and hepatitis C (IFN-alpha(2b)) patients. Chavanpatil, M. D., et al, (Pharm Res, 2007, 24(4):803-10) describe surfactant-polymer nanoparticles as a novel platform for sustained and enhanced cellular delivery of water-soluble molecules. Takeuchi, H., et al, (Adv Drug Deliv Res, 2001, 47(1):39-54) describe mucoadhesive nanoparticulate systems for peptide drug delivery, including liposomes and polymeric nanoparticles. Wong, H. L., et al, (Pharm Res, 2006, 23(7):1574-85) describe a new polymer-lipid hybrid system which has been shown to increase cytotoxicity of doxorubicin against multidrug-resistant breast cancer cells. AMP-N cells and/or ACCS-N and/or pooled ACCS-N are suitable for use in the aforementioned sustained-release formulation methodologies.

In addition, other sustained-release methodologies familiar to skilled artisans, while not specifically described herein, are also suitable for use with AMP-N cells and/or ACCS-N and/or pooled ACCS-N.

Pharmaceutical Compositions—The present invention provides pharmaceutical compositions of AMP-N cells and/or ACCS-N and/or pooled ACCS-N and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

Pharmaceutical compositions useful in the practice of certain embodiments of the invention (i.e. those embodiments utilizing topical administration) include a therapeutically effective amount of an active agent with a pharmaceutically acceptable carrier. Such pharmaceutical compositions may be liquid, gel, ointment, salve, slow release/sustained release formulations or other formulations suitable for administration to treat a nervous system disease, disorder or injury. The pharmaceutical composition comprises a composition of the invention (i.e. AMP-N cells and/or ACCS-N and/or pooled ACCS-N) and, optionally, at least one pharmaceutically acceptable excipient.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits—The invention also provides for an article of manufacture or a kit comprising packaging material and a pharmaceutical composition of the invention contained within the packaging material, wherein the pharmaceutical composition comprises compositions of AMP-N cells and/or ACCS-N and/or pooled ACCS-N. The packaging material comprises a label or package insert which indicates that the AMP-N cells and/or ACCS-N and/or pooled ACCS-N can be used for treating a nervous system disease, disorder or injury.

Exemplary Therapeutic Uses of AMP-N cells and/or ACCS-N and/or pooled ACCS-N

Brain injury is any injury to the brain and can be caused by fracture or penetration of the skull, a disease process, a toxin or infection, or a closed head injury such as rapid acceleration or deceleration of the head.

Traumatic Brain Injuries (TBI) can result from a closed head injury or a penetrating head injury. A closed injury occurs when the head suddenly and violently hits an object but the object does not break through the skull. A penetrating injury occurs when an object pierces the skull and enters brain tissue. Skull fractures occur when the bone of the skull cracks or breaks. A depressed skull fracture occurs when pieces of the broken skull press into the tissue of the brain. A penetrating skull fracture occurs when something pierces the skull, such as a bullet, leaving a distinct and localized injury to brain tissue. Skull fractures can cause cerebral contusion.

Another insult to the brain that can cause injury is anoxia. Anoxia is a condition in which there is an absence of oxygen supply to an organ's tissues, even if there is adequate blood flow to the tissue. Hypoxia refers to a decrease in oxygen supply rather than a complete absence of oxygen, and ischemia is inadequate blood supply, as is seen in cases in which the brain swells. In any of these cases, without adequate oxygen, a biochemical cascade called the ischemic cascade is unleashed, and the cells of the brain can die within several minutes. This type of injury is often seen in near-drowning victims, in heart attack patients, or in people who suffer significant blood loss from other injuries that decrease blood flow to the brain.

All of the above result in neurodegeneration which is the progressive loss of neurons in the brain. Multiple physiological events lead to the neurodegeneration of the brain tissues following a traumatic injury. These events include, for example, cerebral edema, destruction of vascular integrity, increases in the immune and inflammatory response, demyelinization, and lipid peroxidation. Hence, the methods of the instant invention are useful in reducing and/or preventing the physiological events leading to such neurodegeneration. Specifically, the present invention provides methods for reducing or eliminating neuronal cell death (directly or indirectly), edema, ischemia, and enhancing tissue viability following a traumatic injury to the central nervous system.

Spinal Cord Injury—Common causes of spinal cord injury include fractures of the vertebrae, which can damage the spinal cord from the concussive effect of injury due to displaced bony fragments, or damaged blood vessels, or contusion of emerging nerve roots. Dislocation of vertebrae can also cause spinal cord damage; dislocation is often the result of the rupture of an intervertebral disk, and may result in partial or complete severance of the spinal cord. Penetrating wounds can also cause severance or partial severance of the cord. Epidural hemorrhage and spinal subdural hematoma can result in progressive paraparesis due to pressure on the spinal cord. Examples of indirect injury to the spinal cord include damage induced by a blow to the head or a fall on the feet.

Intramedullary injury can be the result of direct pressure on the cord or the passage of a pressure wave through the cord, laceration of the cord by bone, or the rupture of a blood vessel during the passage of a pressure wave through the cord with a hemorrhage into the cord. Intramedullary bleeding and hematoma formation can also be caused by rupture of a weakened blood vessel. Ischemic damage can occur following compression of the anterior spinal artery, pressure on the anastornotic arteries, or damage to major vessels (Gilroy, in Basic Neurology, McGraw-Hill, Inc. New York, N.Y. (1990).

Spinal cord injuries can be divided into two categories, complete injuries and incomplete injuries. It is possible that the classification of the injury may change during recovery. Complete spinal cord injuries are usually characterized by the loss of sensation and motor ability and are generally associated with spinal cord injury caused by bruising, loss of blood to the spinal cord, or pressure on the spinal cord. Cut and severed spinal cords are rare. Generally, complete spinal cord injuries result in total loss of sensation and movement below the site of the injury.

Incomplete spinal cord injuries generally do not result in complete loss of movement and sensation below the injury site. A variety of patterns exist to classify such injuries including 1) anterior cord syndrome which results from damage to the motor and sensory pathways in the anterior areas of the spinal cord. Effects include loss of movement and overall sensation, although some sensations that travel by way of the still intact pathways can be felt; 2) central cord syndrome which results from injury to the center of the cervical area of the spinal cord. The damage affects the corticospinal tract, which is responsible for carrying signals between the brain and spinal cord to control movement. Patients suffering from central cord syndrome experience weakness or paralysis in the arms and some loss of sensory reception. The loss of strength and sensation is much less in the legs than in the arms. Many patients with central cord syndrome spontaneously recover motor function, and others experience considerable recovery in the first six weeks following the injury; 3) Brown-Sequard syndrome results from injury to the right or left side of the spinal cord. On the side of the body where the injury occurred, movement and sensation are lost below the level of the injury. On the side opposite the injury, temperature and pain sensation are lost due to the crossing of these pathways in the spinal cord; 4) injuries to individual nerve cells results in loss of sensory and motor function in the area of the body to which the injured nerve root corresponds. Thus, symptoms from these injuries vary depending on the location and function of the particular nerve root affected; 5) spinal contusions which are the most common type of spinal cord injury. The spinal cord is bruised, not severed, but the consequence is inflammation and bleeding from blood vessels near the injury. A spinal contusion may result in temporary (usually one to two days) incomplete or complete debilitation of the spinal cord or the incomplete or complete debilitation of the spinal cord may be longer term, including a permanent incomplete or complete debilitation of the spinal cord.

A goal of treating spinal cord injury includes promoting motor recovery. Another goal is promoting sensory recovery. Various modalities have been attempted to achieve such motor and sensory recovery, most with only limited success. These studies include application of various growth factors and cytokines to the site of injury as well as transplantation of brain-derived stem cells or healthy spinal cord tissue. Stem cell transplantation therapies are among the most promising therapies currently being studied (see, for example, Cummings, B. J., et al., Neurol Res 2006 28(5):474-81; Pallini, R., et al., Neurosurgery 2005 57(5):1014-25; Cummings, B. J., et al., PNAS USA 2005 102(39):14069-74; Sankar, V. et al., Neuroscience 2003 118(1):11-7).

Degenerative Diseases of the Nervous System

Alzheimer's Disease—Alzheimer's disease (AD), the most common type of dementia, is a neurodegenerative disease characterized by progressive cognitive deterioration together with declining activities of daily living and neuropsychiatric symptoms or behavioral changes. The most obvious early symptom is loss of short-term memory, which usually manifests as minor forgetfulness that becomes steadily more pronounced, with relative preservation of older memories. As the disease progresses, cognitive impairment extends to language, skilled movements, recognition, and functions such as decision-making and planning The pathological process consists primarily of neuronal loss or atrophy, mainly in the temporoparietal cortex, but also in the frontal cortex, together with an inflammatory response to the deposition of amyloid plaques and neurofibrillary tangles.

While the ultimate cause of AD is unknown, genetic factors are known to be important and dominant mutations in three different genes have been identified that account for a much smaller number of cases of familial, early-onset AD. For the more common form of late onset AD, ApoE is the only repeatibly confirmed susceptibility genes for AD.

At autopsy, both amyloid plaques and neurofibrillary tangles are clearly visible by microscopy. At an anatomical level, AD is characterized by gross diffuse atrophy of the brain and loss of neurons, neuronal processes and synapses in the cerebral cortex and certain subcortical regions. This results in gross atrophy of the affected regions.

Current treatment involves acetylcholinesterase inhibitors (i.e. donepezil (Aricept), galantamine (Razadyne) and rivastigmine (Exelon)), the natural extract Gingko Biloba, NMDA antagonists (i.e. memantine (Akatinol, Axura, Ebixa, and Namenda)), and psychosocial intervention (i.e. counseling, psychotherapy, reminiscent therapy, reality orientation, behavioral reinforcements and cognitive rehabilitation training) The compositions and methods of the present invention are effective in treating AD.

Frontotemporal dementia (FTD)—describes a clinical syndrome associated with shrinking of the frontal and temporal anterior lobes of the brain (formerly known as Pick's disease). The current designation of the syndrome groups together Pick's disease, primary progressive aphasia, and semantic dementia as FTD. Some doctors propose adding corticobasal degeneration and progressive supranuclear palsy to FTD and calling the group Pick Complex. As it is defined today, the symptoms of FTD fall into two clinical patterns that involve either (1) changes in behavior, or (2) problems with language. The first type features behavior that can be either impulsive (disinhibited) or bored and listless (apathetic). The second type primarily features symptoms of language disturbance, including difficulty making or understanding speech, often in conjunction with the behavioral type's symptoms. Spatial skills and memory remain intact. There is a strong genetic component to the disease and FTD often runs in families. The compositions and methods of the present invention are effective in treating FTD.

Parkinson's Disease—is caused by the progressive impairment or deterioration of neurons in an area of the brain known as the substantia nigra. When functioning normally, these neurons produce a vital brain chemical known as dopamine. Dopamine serves as a chemical messenger allowing communication between the substantia nigra and another area of the brain called the corpus striatum. This communication coordinates smooth and balanced muscle movement. A lack of dopamine results in abnormal nerve functioning, causing a loss in the ability to control body movements.

Parkinson's disease is currently treated with drugs or, in some cases, surgery. Two general approaches to the treatment of Parkinson's disease with medication are 1) slow the loss of dopamine in the brain and 2) improve the symptoms of Parkinson's disease. Most patients with Parkinson's disease can be adequately treated with medications that alleviate their symptoms. If medications are not sufficiently effective, new, highly effective and safe surgical treatments are also available. Drugs currently available to treat Parkinson's disease include Sinemet (levodopa/carbidopa) Levodopa (also called L-dopa), which is the most commonly prescribed and most effective medication for controlling the symptoms of Parkinson's disease; Symmetrel, which may be helpful for people with mild Parkinson's disease, but it often causes significant side-effects including confusion and memory problems; Anticholinergics (Artane, Cogentin) are used to restore the balance between the two brain chemicals, dopamine and acetylcholine, by reducing the amount of acetylcholine. These medications, however, can impair memory and thinking, especially in older people; therefore, they are rarely used today; Eldepryl and Deprenyl are two names for the same drug. They work by helping to conserve the amount of dopamine available by preventing the dopamine from being destroyed. While controversial, there is some evidence that this drug may slow the progression of Parkinson's disease, particularly early in the course of the disease. This drug is well-tolerated by most people, so many experts recommend using it despite the controversies. Tasmar, Comtan (COMT Inhibitors). When COMT is blocked, dopamine can be retained and used more effectively, reducing Parkinson's symptoms. COMT inhibitors can also increase the effectiveness of levodopa.

Surgical options include deep brain stimulation which is a method to inactivate the parts of the brain that cause Parkinson's disease and its associated symptoms without purposefully destroying the brain. In deep brain stimulation, electrodes are placed in the globus pallidus. The electrodes are connected by wires to a type of pacemaker device (called an impulse generator, or IPG) that is implanted under the skin of the chest. Once activated, the device sends continuous electrical pulses to the target areas in the brain, blocking the impulses that cause tremors. This has the same effect as thalamotomy or pallidotomy surgeries without actually destroying parts of the brain. The stimulation can be turned on or off by the patient with a hand-held magnet or an access control device. The compositions and methods of the present invention are effective in treating Parkinson's disease.

Huntington's disease (HD)—results from genetically programmed degeneration of neurons in certain areas of the brain. This degeneration causes uncontrolled movements, loss of intellectual faculties, and emotional disturbance. HD is a familial disease, passed from parent to child through a mutation in the normal gene. Each child of an HD parent has a 50-50 chance of inheriting the HD gene. If a child does not inherit the HD gene, he or she will not develop the disease and cannot pass it to subsequent generations. A person who inherits the HD gene will sooner or later develop the disease. Some early symptoms of HD are mood swings, depression, irritability, learning new things, remembering a fact, or making a decision. As the disease progresses, concentration on intellectual tasks becomes increasingly difficult and the patient may have difficulty feeding himself and swallowing. The rate of disease progression and the age of onset vary from person to person.

Current treatments for HD address the symptoms, not the underlying disease itself. For example, tranquilizers such as clonazepam (Klonopin) and antipsychotic drugs such as haloperidol (Haldol) and clozapine (Clozaril) are used to help control movements, violent outbursts and hallucinations. Other drugs such as fluoxetine (Prozac, Sarafem), sertraline (Zoloft) and nortriptyline (Aventyl, Pamelor), are used to help control depression and the obsessive-compulsive behaviors often exhibited by some HDS patients. Medications such as lithium (Eskalith, Lithobid) can help control extreme emotions and mood swings. Side effects from the drugs used to treat the symptoms of HD include hyperexcitability, fatigue and restlessness. In some instances, antipsychotic drugs may cause side effects that mimic the signs of Parkinson's disease, including involuntary twitching of the face and body (tardive dyskinesia). Because HD can impair speech therapy is often prescribed. The compositions and methods of the present invention are effective in treating HD.

Motor Neuron Diseases

Amyotrophic lateral sclerosis (ALS)—sometimes called Lou Gehrig's disease, is a progressive, fatal neurodegenerative disease caused by the degeneration of motor neurons. ALS is marked by gradual degeneration of the neurons in the CNS that control voluntary muscle movement. The disorder causes muscle weakness and atrophy throughout the body. In ALS, both the upper motor neurons and the lower motor neurons degenerate or die, ceasing to send messages to muscles. Unable to function, the muscles gradually weaken and atrophy. Eventually, the brain completely loses its ability to initiate and control voluntary movement. The disease does not necessarily debilitate the patient's mental functioning in the same manner as Alzheimer's disease or other neurological conditions do. Instead, those suffering advanced stages of the disease may retain the same memories, personality, and intelligence they had before its onset.

Current treatment for ALS is very limited. One drug, riluzole (Rilutek®) is believed to reduce damage to motor neurons and prolong survival by several months, mainly in those with difficulty swallowing. Another drug, gabapentin (Neurotin®), is a seizure medication and is believed to work to reduce glutamate production. It may be beneficial to some ALS patients. The compositions and methods of the present invention are effective in treating ALS.

Spinal muscular atrophy (SMA)—is a genetic, motor neuron disease caused by progressive degeneration of motor neurons in the spinal cord. The disorder causes weakness and wasting of the voluntary muscles. Weakness is often more severe in the legs than in the arms. The childhood SMAs are all autosomal recessive diseases. This means that they run in families and more than one case is likely to occur in siblings or cousins of the same generation. There are many types of SMA; some of the more common types are as follows: SMA type I, also called Werdnig-Hoffmann disease, is evident before birth or within the first few months of life. There may be a reduction in fetal movement in the final months of pregnancy. Symptoms include floppiness of the limbs and trunk, feeble movements of the arms and legs, swallowing and feeding difficulties, and impaired breathing. Affected children never sit or stand and usually die before the age of 2. Symptoms of SMA type II usually begin between 3 and 15 months of age. Children may have respiratory problems, floppy limbs, decreased or absent deep tendon reflexes, and twitching of arm, leg, or tongue muscles. These children may learn to sit but will never be able to stand or walk. Life expectancy varies. Symptoms of SMA type III (Kugelberg-Welander disease) appear between 2 and 17 years of age, and include abnormal manner of walking; difficulty running, climbing steps, or rising from a chair; and slight tremor of the fingers. Kennedy syndrome or progressive spinobulbar muscular atrophy may occur between 15 and 60 years of age. Features of this type may include weakness of muscles in the tongue and face, difficulty swallowing, speech impairment, and excessive development of the mammary glands in males. The course of the disorder is usually slowly progressive. Congenital SMA with arthrogryposis (persistent contracture of joints with fixed abnormal posture of the limb) is a rare disorder. Manifestations include severe contractures, curvature of the spine, chest deformity, respiratory problems, an unusually small jaw, and drooping upper eyelids.

There are no drugs for treating SMA, although much can be done to manage patients medically, in particular, managing respiratory, nutritional and rehabilitation care. There are several drugs currently under investigation, including butyrates, valproic acid, hydroxyurea and riluzole. The compositions and methods of the present invention are effective in treating SMAs.

Progressive bulbar palsy—is a disorder in which the nerves controlling the muscles of chewing, swallowing, and talking are affected, making these functions increasingly difficult. Because swallowing is difficult, food or saliva is often inhaled (aspirated) into the lungs, causing choking or gagging and increasing the risk of pneumonia. Death, which is often due to pneumonia, usually occurs 1 to 3 years after the disorder begins. Most treatment is directed to managing symptoms. Riluzole may be effective in certain patients. The compositions and methods of the present invention are effective in treating progressive bulbar palsy.

Primary Lateral Sclerosis and Progressive Pseudobulbar Palsy—are rare, slowly progressive variants of amyotrophic lateral sclerosis. Primary lateral sclerosis affects mainly the arms and legs, and progressive pseudobulbar palsy affects mainly the muscles of the face, jaw, and throat. Emotions may be changeable. Inappropriate emotional outbursts are common. In both disorders, severe stiffness accompanies muscle weakness. The disorders usually progress for several years before total disability results. Most treatment is directed to managing symptoms. Riluzole may be effective in certain patients. The compositions and methods of the present invention are effective in treating primary lateral sclerosis and progressive pseudobulbar palsy.

Post-polio syndrome—Some people who have had polio may develop tired, painful, and weak muscles 15 years or more after their recovery from polio. Sometimes muscle tissue also wastes away, suggesting a reactivation of the polio infection. There are no specific treatments for post-polio syndrome. Most treatments are directed to treating symptoms. The three primary symptoms that are treated with medication are weakness of muscle, fatigue (individual muscle and generalized), and pain, i.e., post-polio pain, overuse pain, bio-mechanical pain, and bone pain. The compositions and methods of the present invention are effective in treating post-polio syndrome.

Demyelinating Diseases

A. Acquired

Multiple Sclerosis (MS)—is an unpredictable disease of the central nervous system that can range from relatively benign to somewhat disabling to devastating, as communication between the brain and other parts of the body is disrupted. Many investigators believe MS to be an autoimmune disease in which the nerve-insulating myelin is attacked. Most people experience their first symptoms of MS between the ages of 20 and 40 and the initial symptom of MS is often blurred or double vision, red-green color distortion, or even blindness in one eye. Most patients experience muscle weakness in their extremities and difficulty with coordination and balance. The symptoms may be severe enough to impair walking or even standing and in the worst cases, can produce partial or complete paralysis. Most people with MS also exhibit paresthesias. Speech impediments, tremors, and dizziness are other frequent complaints and occasionally, patients experience hearing loss. Approximately half of all people with MS experience cognitive impairments such as difficulties with concentration, attention, memory, and poor judgment, but such symptoms are usually mild. Several forms of MS exist including Benign MS, Relapsing Remitting MS (the most common form), Secondary Progressive MS, Primary Progressive MS, Malignant MS (Marburg variant) and Chronic MS.

Several drugs are available to treat MS, including interferon beta-1b (Betaseron®), interferon beta-1a (Avonex®), high dose/frequency interferon beta-1a (Rebif®), glatiramer (Copaxone®), mitoxantrone (Novantrone®) and corticosteroids. The compositions and methods of the present invention are effective in treating MS.

Balo's concentric sclerosis (Balo Disease)—is a rare and progressive variant of MS. It usually first appears in adulthood, but childhood cases have also been reported. While MS typically is a disease that waxes and wanes, Balo concentric sclerosis is different in that it tends to be rapidly progressive. Symptoms may include headache, seizures, gradual paralysis, involuntary muscle spasms, and cognitive loss. The disease is characterized by bands of intact myelin (the sheath of fatty substances surrounding nerve fibers) alternating with rings of loss of myelin (demyelination) in various parts of the brain and brain stem. Symptoms may progress rapidly over several weeks or more slowly over two to three years. The same drugs used to treat MS are used to treat Balo Disease. The compositions and methods of the present invention are effective in treating Balo's concentric sclerosis.

Acute disseminating encephalomyelitis (ADEM)—is characterized by a brief but intense attack of inflammation in the brain and spinal cord that damages myelin. It often follows viral infection, or less often, vaccination for measles, mumps, or rubella. The symptoms of ADEM come on quickly, beginning with encephalitis-like symptoms such as fever, fatigue, headache, nausea and vomiting, and in severe cases, seizures and coma. It may also damage white matter, leading to neurological symptoms such as visual loss in one or both eyes, weakness even to the point of paralysis, and difficulty coordinating voluntary muscle movements. ADEM is sometimes misdiagnosed as a severe first attack of MS. However, ADEM usually has symptoms of encephalitis (such as fever or coma), as well as symptoms of myelin damage (visual loss, paralysis). In addition, ADEM usually consists of a single episode or attack, while MS features many attacks over the course of time. Children are more likely than adults to have ADEM. The compositions and methods of the present invention are effective in treating ADEM.

Neuromyelitis Optica (Devic's Disease)—is an inflammatory disease of the CNS in which there are episodes of inflammation and damage to the myelin that almost exclusively affect the optic nerves and spinal cord. It usually causes temporary blindness, occasionally permanent, in one or both eyes. It can also lead to varying degrees of weakness or paralysis in the legs or arms, loss of sensation, and/or bladder and bowel dysfunction from spinal cord damage. The compositions and methods of the present invention are effective in treating neuromyelitis optica.

Transverse myelitis (TM)—is a neurologic syndrome caused by inflammation of the spinal cord. TM is uncommon but not rare. The term myelitis is a nonspecific term for inflammation of the spinal cord; transverse refers to involvement across one level of the spinal cord. It occurs in both adults and children. The compositions and methods of the present invention are effective in treating transverse myelitis.

B. Hereditary

Leukodystrophies—refers to a group of disorders characterized by progressive degeneration of the white matter of the brain. The leukodystrophies are caused by imperfect growth or development of the myelin sheath. Myelin is a complex substance made up of at least ten different molecules. Each of the leukodystrophies is the result of a defect in the gene that controls the production or metabolism of one (and only one) of the component molecules of myelin. The different types of leukodystrophy including adrenoleukodystrophy, metachromatic leukodystrophy, Krabbe disease, Pelizaeus-Merzbacher disease, Canavan disease, childhood ataxia with central hypomyelination (CACH or vanishing white matter disease), Alexander disease, Refsum disease and cerebrotendineous xanthomatosis. The compositions and methods of the present invention are effective in treating leukodystrophies.

Peripheral Diseases

Peripheral neuropathy, in its most common form, causes pain and numbness in the hands and feet. The pain typically is described as tingling or burning, while the loss of sensation often is compared to the feeling of wearing a thin stocking or glove. Peripheral neuropathy can result from such problems as traumatic injuries (i.e. axotomy distal to the dorsal root ganglia) or surgical incisions, compression of nerves (i.e. Tic douloureux), post-herpetic infections (i.e. herpes zoster infection), HIV infection, metabolic problems (i.e. diabetes), hereditary sensory and autonomic neuropathies, exposure to toxins (i.e. neurotoxic chemotherapy induced peripheral neuropathy), and drugs (i.e. antiretroviral drugs). In many cases, peripheral neuropathy symptoms improve with time, especially if it is caused by an underlying condition that can be resolved or at least managed. Medications initially designed to treat other conditions, such as epilepsy and depression, are often used to reduce the painful symptoms of peripheral neuropathy. However, treatment options are still limited. The compositions and methods of the present invention are effective in treating peripheral neuropathy.

The term “nervous system injury” refers to any injury of the nervous tissue and can be caused by fracture or penetration of the skull or vertebra (such as in the case of a vehicle accident, fall or gunshot wound resulting in damage to the brain or spinal cord), a disease process (neurotoxins, infections, tumors, metabolic abnormalities, genetic abnormalities, degenerative nerve diseases, etc.), a closed head injury such as rapid acceleration or deceleration of the head (i.e. Shaken Baby Syndrome) causing injury to the brain, or an injury or disease affecting the peripheral nerves. Such injuries can have devastating lifelong effects on physical and mental functioning.

Skilled artisans will recognize that any and all of the standard methods and modalities for treating a nervous system disease, disorder or injury currently in clinical practice and clinical development are suitable for practicing the methods of the invention. Routes of administration, formulation, co-administration with other agents (if appropriate) and the like are discussed in detail elsewhere herein.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Preparation of AMP Cell Compositions.

Amnion epithelial cells were dissociated from starting amniotic membrane using the dissociation agent PXXIII. The average weight range of an amnion was 18-27 g. The number of cells recovered per g of amnion was about 10-15×10⁶.

Method of obtaining selected AMP cells—Amnion epithelial cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in basal medium supplemented with human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2: Creation of AMP-N Cells

AMP cells described above were cultured according to a proprietary, multistep culture process that resulted in the creation of a novel cell type called AMP-N cells. AMP-N cells were characterized as described below.

### Example 3: Cell Surface Marker Characterization of AMP-N Cells

The AMP-N cells which result from the proprietary, multistep culture process exhibit a unique combination of cell markers that has yet to be described in a cell. The AMP-N cells exhibit neuronal-like markers in that they express near-neuronal levels of β-tubulin-III and MAP2. In contrast, AMP cells which are not treated in the proprietary, multistep culture process exhibit either very low or no expression of these neuronal markers. Importantly, AMP-N cells have been shown to be different from neurons in the lack of expression of other neuronal markers. Specifically, neurons exhibit a high expression of surface markers such as PSA-NCAM and NFM, whereas AMP-N cells and AMP cells exhibit a low expression of these markers. AMP-N cells (and AMP cells) were also shown to express high levels of the glial cell markers GFAP and a-S100, both of which are expressed at very low levels in neurons. Finally, AMP-N cells and AMP cells were analyzed for the expression of the stem cell marker SSEA-4. AMP cells are positive for this marker, as would be expected for this cell type, while the level of this stem cell marker went down in the AMP-N cells, indicating that they are losing their “stem-ness” when they are treated in the proprietary, multistep culture process. Neurons, which are terminally differentiated cells, do not express stem cell markers such as SSEA-4.

Thus, Applicants' have created a novel cell type exhibiting a unique combination of markers that is comprised of some neuronal markers (i.e., β-tubulin-III and MAP2), but not others (i.e., PSA-NCAM and NFM), some glial markers (i.e., GFAP and α-S100), and some AMP cell markers (i.e., SSEA-4).

### Example 4: Comparison of Gene Expression Profiles of AMP-N Cells Relative to Undifferentiated AMP Cells

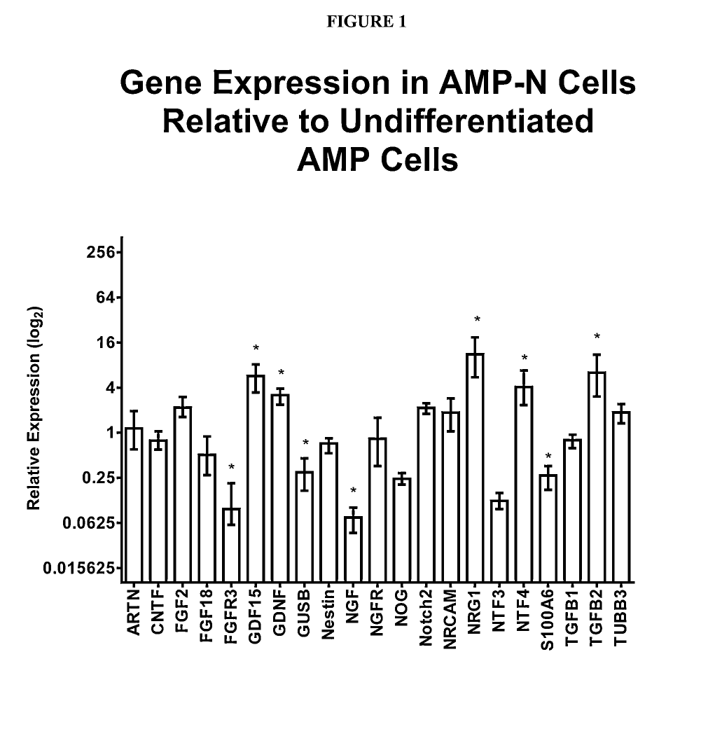

Quantitative RT-PCR was used to compare the expression of 28 genes in AMP-N cells and undifferentiated AMP cells. In this experiment, identical expression in the two cell types would result in a relative expression=1 (indicated by the dashed line in FIG. 1). Genes with a higher level of expression in AMP-N cells relative to AMP cells have a relative expression greater than 1 while genes with a lower level of expression in AMP-N cells have a relative expression less than 1. Genes with a significant difference (p<0.05) in expression greater than threefold are indicated by asterisks (FIG. 1). Expression results and statistical analysis were generated by randomization analysis using REST 2009 software and represent the mean of three replicates for each cell type (M. W. Pfaffl et al. Nucleic Acids Research 2002 May 1; 30(9): E36). Error bars represent the 95% confidence interval. Expression of the 18S RNA was used as the reference for each sample. No expression of FGF4,GFAP, IL-10, NeuroD1, S100B, SHH, Sox2, or Sox10 was observed in either cell type.

### Example 5: Comparison of Gene Expression Profiles of AMP-N Cells Relative to Neurons

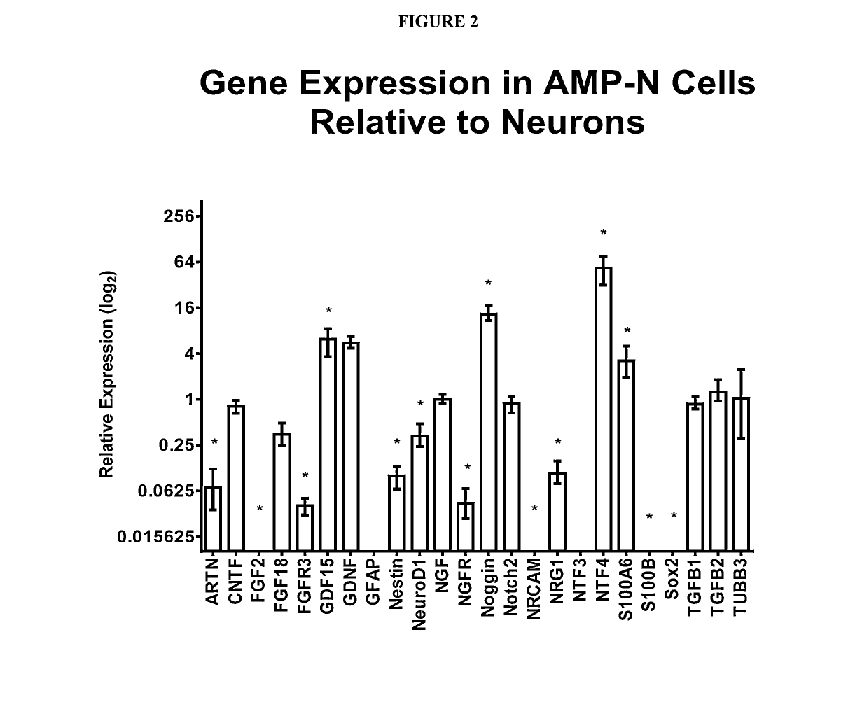

Quantitative RT-PCR was used to compare the expression of 28 genes in AMP-N cells and human neurons (Sciencell #1520-10). In this experiment, identical expression in the two cell types would result in a relative expression=1 (indicated by dashed line in FIG. 2). Genes with a higher level of expression in AMP-N cells relative to neurons have a relative expression greater than 1 while genes with a lower level of expression in AMP-N cells have a relative expression less than 1. Genes with a significant difference (p<0.05) in expression greater than threefold are indicated by asterisks (FIG. 2). FGF2, GFAP, NRCAM, S100B, and Sox2 showed extremely low levels of expression in AMP-N cells, resulting in means not able to be adequately represented in this Figure. Expression results and statistical analysis were generated by randomization analysis shown using REST 2009 software and represent the mean of three replicates for each cell type (M. W. Pfaffl et al. Nucleic Acids Research 2002 May 1; 30(9): E36). Error bars represent the 95% confidence interval. Expression of the 18S RNA was used as the reference for each sample. No expression of FGF4, IL-10, Sox10, or SHH was observed in either AMP-N cells or neurons.

### Example 6: Generation of ACCS-N from AMP-N Cells

The AMP-N cells described above are particularly well-suited for the production of a conditioned medium having a unique combination of secreted proteins that may be useful in treating diseases, disorders and injuries of the nervous system. This conditioned medium is made by culturing the AMP-N cells in the proprietary, multi-step process and then collecting the conditioned medium. In some cases, multiple collections of conditioned medium may be pooled together to create a more consistent lot of the conditioned medium with respect to levels of secreted proteins.

### Example 7: Characterization of ACCS-N

ACCS-N is characterized for its secreted protein profile using standard ELISA, antibody array and mass spectroscopy technology.

### Example 8: Generation of Sustained-Release Compositions

Sustained-release compositions of AMP-N cells and/or ACCS-N and/or pooled ACCS-N are produced by combining AMP-N cells and/or ACCS-N and/or pooled ACCS-N compositions with any of the sustained-release formulation technologies described herein (see Detailed Description) or otherwise familiar to skilled artisans.

### Example 9: Use of AMP-N cells and/or ACCS-N and/or pooled ACCS-N in animal models of nervous system diseases and disorders.

Alzheimer's disease-Wenk, G. L. (Behav Brain Res 1993 57(2):117-22) describe a model of Alzheimer's disease which is based upon the assumption that the destruction of basal forebrain cholinergic neurons by injection of a neurotoxin, such as ibotenic acid, is sufficient to reproduce the cognitive impairments associated with Alzheimer's disease. AMP-N cells and/or ACCS-N and/or pooled ACCS-N are tested in this model of Alzheimer's disease.

Parkinson's disease-Arai, N., et al. (Brain Res 1990 515(1-2):57-63) describe an animal model for parkinsonism in which C57 black mice are treated with MPTP. The authors conclude that this model is a suitable model for studying Parkinson's disease. AMP-N cells and/or ACCS-N and/or pooled ACCS-N are tested in this model of Parkinson's disease.

Huntington's disease—Reddy, P. H., et al., (Nat Genet 1998 20(2):198-202) describe an experimental model for Huntington's disease in which transgenic mice were created that exhibited widespread expression of the full-length human Huntington's disease cDNA with either 16, 48 or 89 CAG repeats. The mice with 48 or 89 CAG repeats manifested progressive behavioral and motor dysfunction with neuron loss and gliosis in the striatum, cerebral cortex, thalamus and hippocampus. The authors conclude that this model is a clinically relevant model for Huntington's disease pathogenesis. AMP-N cells and/or ACCS-N and/or pooled ACCS-N are tested in this model of Huntington's disease.

Amyotrophic Lateral Sclerosis—Pioro, E. P. and Mitsumoto, H. (Clin Neurosci 1995-1996 3(6):375-85) describe four animal models of ALS. AMP-N cells and/or ACCS-N and/or pooled ACCS-N are tested in this model of ALS.

Spinal muscular atrophy—Monani, U. R., et al., Hum Mol Genet 2000 9(16):2451-2457) review animal models of spinal muscular atrophy. AMP-N cells and/or ACCS-N and/or pooled ACCS-N are tested in this model of spinal muscular atrophy disease.

Multiple sclerosis—Peiris, M., et al., (J Neurosci Methods 2007 Mar. 30 epub) describe an animal model of experimental autoimmune encephalomyelitis (EAE) in C57BL/6 mice useful for the characterization of intervention therapies to treat Multiple sclerosis. AMP-N cells and/or ACCS-N and/or pooled ACCS-N are tested in this model of Multiple Sclerosis.

Peripheral neuropathy—Fricker, et al, (Neurodegen Dis 2008, 5:72-108) provide an extensive review of experimental peripheral neuropathy, including a comprehensive outline of numerous animal models useful in studying all types of peripheral neuropathy. For example, animal models for inherited (HNPP, CMT1A, CMT1B, DSS, CMT1X, CMT4B1), infectious (Leprosy, HIV), immune (GBS), diabetic (Type I, Type II), injury (transient nerve crush, chronic constriction injury, partial nerve ligation, spinal nerve ligation, spared nerve injury), and chemotherapy (i.e. cisplatin)-induced neuropathies are provided. AMP-N cells and/or ACCS-N and/or pooled ACCS-N are tested in these models of peripheral neuropathy.

### Example 10: Use of AMP-N cells and/or ACCS-N and/or pooled ACCS-N in animal models of nervous system injury.

Traumatic Brain Injury (TBI)—AMP-N cells and/or ACCS-N and/or pooled ACCS-N are tested in a rat model of Penetrating Ballistic-like Brain Injury (PBBI) (Williams, A. J., 2005, “Characterization of a New Rat Model of Penetrating Ballistic Brain Injury”, J Neurotrauma 22; 3:313-331) to test their neuroprotective potential. The AMP-N cells and/or ACCS-N and/or pooled ACCS-N are injected in rats immediately following right frontal PBBI or sham PBBI surgery by ipsilateral i.c.v. administration. PBBI controls will receive i.c.v. injection of PBS, control medium, or conditioned medium.

Spinal Cord Injury (SCI)—AMP-N cells and/or ACCS-N and/or pooled ACCS-N are tested in a mouse model of SCI. Nine days after contusion injury (see, for example, Constantini, S., and Young, W. (1994), J. Neurosurg. 80, 97-111; Anderson, A. J., et al., Journal of Neurotrauma 21 (12), 1831-1846 for animal models), AMP-N cells and/or ACCS-N and/or pooled ACCS-N are administered into two different sites, the injury epicenter or the adjacent nervous tissue parenchyma.

Analysis—Open field locomotor testing is the standard of the spinal cord injury field, because it is the only task which allows the investigator to evaluate a full spectrum of potential recovery, from complete paralysis (a 0 on the BBB or BMS) to fully normal locomotion (a 21 on the BBB, and a 9 on the BMS). However, these are nonlinear (ordinal) scales. It is not a direct comparison of the number of points of change that is relevant, but rather what these points represent in terms of specifically recovered locomotor function. In these tasks, animals move freely in a circular area for a four minute testing period while two investigators blinded to experimental group observe and rank the animals based on specific criteria on each testing scale. In the case of the range of recovery for the animals in this pilot, the key criterion scored by the investigators involves the ability to achieve consistent stepping and complete coordinated “passes”. Each unbroken series of movements across the arena greater than three body lengths is defined as a pass. Normal animals make one hindlimb step for each forelimb step; missed steps decrease the score an animal achieves on the scale.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A composition comprising AMP-N cells.

2. The composition of claim 1 wherein the AMP-N cells express high levels of SSEA-4, GFAP, α-S100 and low levels of β-tubulin III, MAP2, PSA-NCAM and NFM.

3. The composition of claim 1 which is a pharmaceutical composition.

4. The composition of claim 3 wherein the pharmaceutical composition is contained in an article of manufacture, wherein the article of manufacture comprises the pharmaceutical composition, packaging material, and instructions for use of the pharmaceutical composition.

5. A composition comprising ACCS-N.

6. The composition of claim 5 which is a pharmaceutical composition.

7. The composition of claim 6 wherein the pharmaceutical composition is contained in an article of manufacture, wherein the article of manufacture comprises pharmaceutical composition, packaging material, and instructions for use of the pharmaceutical composition to treat a nervous system disease, disorder or injury.

8. The composition of claim 6 which is formulated for sustained-release.

9. The composition of claim 5 which is pooled ACCS-N.

10. A method for treating a nervous system disease, disorder or injury comprising administering to a subject a therapeutically effective dose of a composition selected from the group consisting of AMP-N cells, ACCS-N and pooled ACCS-N.
